(19) United States
(12) Patent Application Publication
(10) Pub. No.: US 2011/0268813 A1
(43) Pub. Date: Nov. 3, 2011

(54) PREVENTING LIVER INJURY AND IMPROVING LIVER FUNCTION EFFECTS OF ENA-ACTIMINERAL RESOURCES

(75) Inventors: Sung Yong Hwa, Jinju-si (KR); Kyu Shik Jeong, Daegu (KR); Jin Kyu Park, Masan-si (KR); Jung Youn Han, Daegu (KR)

(73) Assignee: Sung Yong HWA, Jinju-si, Gyeongsangnam-do (KR)

(21) Appl. No.: 12/680,741

(22) PCT Filed: Sep. 17, 2009
(86) PCT No.: PCT/KR09/05284

§ 371 Date: Mar. 29, 2010

(30) Foreign Application Priority Data
Sep. 18, 2008 (KR) 10-2008-0091491
Apr. 2, 2009 (KR) 10-2009-0028394

(51) Int. Cl. A61K 35/64; A61P 1/16

(52) U.S. Cl. 424/538

(57) ABSTRACT

Disclosed is a composition with anti-oxidative, anti-aging and liver-function improvement activities containing ENA actimineral resource A activated water. More specifically, disclosed are a pharmaceutical composition and a health food or health supplement containing, as an active ingredient, an alkaline ENA actimineral resource A activated water prepared from Sepia bone and red algae powders, for preventing aging due to inhibitory activity on decrease in serum vitamin C, or preventing liver damage or improving liver functions due to inhibitory activity on damage, apotosis or necrosis of hepatic cells. The composition inhibits a decrease in in vivo serum vitamin C, to inhibit aging and prevents damage, apotosis or necrosis of hepatic cells, and fundamentally protects liver cells to inhibit damage, apotosis or necrosis of hepatic cells by aging in the liver and improve liver function.

### BACKGROUND OF THE INVENTION

1. Field of the Invention

The present invention relates to a composition with anti-oxidative, anti-aging and liver-function improvement activities containing ENA actimineral resource A activated water. More specifically, the present invention relates to a pharmaceutical composition, and a health food or a health supplement containing ENA actimineral resource A activated water as an active ingredient for preventing aging due to inhibitory activity on decrease in serum vitamin C, or preventing liver damage or improving liver function due to inhibitory activity on damage, apotosis or necrosis of hepatic cells.

2. Description of the Related Art

People undergoing rapid development are exposed to a variety of external stress. Stress is inevitable and has an effect on the progression of general aging of the human body. Aging is not a disease, but an unavoidable natural biological phenomenon. Inherent human desire for longevity has brought about a great deal of research to inhibit aging. Furthermore, it is not too much to say that people suffer from gradually increased stress. In accordance with much attention to improvement in quality of life along with the recent well-being trend, a great deal of search on an anti-oxidative material with anti-aging activity comes into the spotlight.

Senescence marker protein (SMP 30) is an aging marker protein with a mass of 34 kDa, was first found in the liver of rats, and is reportedly expressed in rapidly decreased amounts with the progression of aging. This decrease behavior was known to be irrelevant to decreased production of androgen hormone in male rats due to aging (Fujita T., Biochem Biophys Res Commun. 1999 Jan 8; 254 (1):1-4, Mori T et al., Pathology International 2004; 54; 167-1737).

SMP 30 is found to prevent apotosis and necrosis of cells and thus inhibit physical aging. The main mechanisms of SMP 30 are as follows. SMP 30 acts as gluconolactonase associated with synthesis of vitamin C and thus plays an important role in in vivo vitamin C biosynthesis. SMP 30 maintains homeostasis between intracellular and extracellular calcium ions, a signaling molecule, which plays an important role in apotosis and necrosis of cells. In addition, SMP 30 by itself acts as an anti-oxidant protein which destroys active oxygen and radicals harmful to the body to prevent physical aging and apotosis and necrosis of cells. Accordingly, SMP 30 protein deficiency is known to cause vitamin C deficiency and promote aging in animals, as compared to animals having normal SMP 30 proteins.

The increasing elderly population causes rapid development of longevity-associated industries including health supplements, hormone preparations, anti-oxidative medicines, and living and function-supplementary means for the elderly. About 90% of a variety of modem diseases is found to be caused by active oxygen. In this regard, placenta injection with various activities such as skin care and inhibition of menopausal disorder and anti-aging is currently attracts much attention and placenta is found to exhibit removal activity of active oxygen and thus contribute to the treatment of 90% of modern diseases. That is, placenta is known to treat almost all diseases owing to autonomic nerve controlling activity, endocrine controlling activity and immune revitalization activity. However, effects of various ingredients of placenta have not yet researched, and ingredients and effects of food are not marked on food, unlike medicines, since marking thereof is not a legally compulsory regulation. There is no ground supporting the assertion that hormones for anti-aging practically exhibit anti-aging effects and use of these hormones is thus not recommended.

Korean Patent No. 10-0463825 discloses a method for preparing ENA actimineral resource A activated water and a composition for preventing and alleviating osteoporosis using the same. The preventive or inhibitory activities on physical aging of ENA actimineral resource A, as a natural material, which inhibits damage of liver cells and decrease of serum vitamin C and thus inhibits aging of mammals, are not known to date.

### SUMMARY OF THE INVENTION

Therefore, the present invention has been made in view of the above problems, and it is one object of the present invention to provide a pharmaceutical composition and a health food or a health supplement containing ENA actimineral resource A activated water as an active ingredient for preventing aging due to inhibitory activity on decrease in serum vitamin C, or preventing liver damage or improving liver function due to inhibitory activity on damage, apotosis or necrosis of hepatic cells.

In accordance with the present invention, the above and other objects can be accomplished by the provision of a pharmaceutical composition and a health food or a health supplement containing ENA actimineral resource A activated water as an active ingredient for preventing aging due to inhibitory activity on decrease in serum vitamin C, or preventing liver damage or improving liver function due to inhibitory activity on damage, apotosis or necrosis of hepatic cells.

The ENA actimineral resource A activated water inhibits a decrease in in vivo serum vitamin C, to inhibit aging and prevent damage, apotosis or necrosis of hepatic cells.

The ENA actimineral resource A activated water inhibits damage, apotosis or necrosis of hepatic cells to prevent liver damage or improve liver function.

### DETAILED DESCRIPTION OF THE INVENTION

Hereinafter, the present invention will be described in detail.

In accordance with the present invention, provided is an alkaline aqueous solution providing natural minerals prepared by purifying, as main ingredients, naturally edible algae, i.e., laver (Porphyra tenera), agar (Gelidium amansii), sea string (Gracilaria verrucosa), Nemalion vermiculare, Grateloupia filicina, Gigartina tenella, Ceramium kondoi, floridean starch, and Sepia bone (Sepia esculenta). The substance obtained by the method disclosed in Korean Patent No. 463,825 is referred to as “ENA actimineral resource A activated water” and is prepared with reference to the following method.

As used herein, the term “naturally edible algae” refers to an edible 100% vegetarian red algae which contains phycoerythrin as well as chlorophyll and is thus red or violet in color. The algae generally has a multi-cellular thread or leaf shape, is typically found in the sea and includes level, agar, turner (Gloiopeltis tenax), and the like. In addition, the sepia bone is prepared by drying white bone collected from the center of cuttlefish.

The preparation of minerals using red algae or edible cuttlefish is carried out by completely washing red algae or edible cuttlefish, sufficiently drying the same, followed by calcination at 1,000 to 2000° C. for one hour. Only the mineral, inorganic material is left behind after bacteria or impurities are completely combusted by calcination and thus removed. The mineral is completely cooled to ambient temperature and then micro-powderized using a grinder.

Then, the micro-powder mineral containing the calcinated sepia bone and red algae is dissolved in water. Preferably, an ionic solution is prepared by breaking the mineral solution at 80 to 100° C. with 10 rpm or higher of head drop using a water lifting pump for one hour or longer. The ionic solution thus obtained is precipitated and then filtered. More specifically, the ionic solution is allowed to stand for 15 to 35 hours to naturally precipitate a mineral sludge and only the resulting clear supernatant is filtered through a filter, to prepare alkaline mineral activated water.

Effects of the ENA actimineral resource A activated water on difference in survival rate and weight variations, animal skeleton variations and physical conversion were confirmed. More specifically, for example, the ENA actimineral resource A activated water was administered in various concentrations of 0%, 5% and 10% for 18 weeks to 18-week old SMP 30-knockout mice to which vitamin C was administered, and 26-week old and 46-week old SMP 30-knockout mice to which vitamin C was not administered, variations observed by the naked eye and variations in weight and survival rate over the test period were monitored and all subjects were subjected to necropsy after the test period.

As a result, with respect to groups to which vitamin C was administered, there can be observed no difference in weight and survival rate variations between the group to which the ENA actimineral resource A activated water, a test material, was administered, and the group to which the ENA actimineral resource A activated water was not administered (See FIG. 1).

On the other hand, with respect to difference in weight and survival rate variations between groups to which vitamin C was not administered, the group to which the ENA actimineral resource A activated water was administered exhibited significantly rapid decreased weight than a mean weight, according to levels of ENA actimineral resource A activated water and survival rate of 0%. That is, there is a significant difference between the groups according to concentrations of ENA actimineral resource A activated water (See FIGS. 2 to 4).

In addition, as a result of tests to confirm effects of naturally-derived aging symptoms on animal skeleton variations and physical conversion by X-ray irradiation, all groups to which vitamin C was administered did not suffer from scorbutic osteogenic disorders, whereas all groups to which vitamin C was not administered suffered from scorbutic osteogenic disorders (See FIG. 5).

Whether or not the ENA actimineral resource A activated water inhibits a decrease in in vivo serum vitamin C and thus inhibits aging and damage, apotosis or necrosis of hepatic cells was confirmed.

First, as a result of tests confirming effects of the ENA actimineral resource A activated water on serum vitamin C was observed only in groups to which vitamin C was administered, whereas serum vitamin C was not observed only in groups to which vitamin C was not administered. More specifically, for groups to which vitamin C was administered, a group to which the ENA actimineral resource A activated water was administered, exhibited a statistically significantly higher total vitamin C level in the serum, as compared to a control group, to which only an excipient was administered. In addition, it can be seen from a graph showing a ratio of reduced vitamin C to oxidized vitamin C that the group to which the ENA actimineral resource A activated water was administered, exhibited an increased ratio of reduced vitamin C to oxidized vitamin C, depending on concentrations of activated water, as compared to the excipient control group. This behavior indicates that the ENA actimineral resource A activated water inhibits in vivo vitamin C oxidation due to anti-aging function and thus prevents aging (see FIGS. 6 and 7).

Meanwhile, the ENA actimineral resource A activated water inhibits damage, apotosis or necrosis of hepatic cells and thus prevents liver damage or improves liver function. More specifically, for example, in a male SMP 30-knockout mouse aging model at various ages in weeks, with respect to groups to which vitamin C is administered, and groups to which vitamin C is not administered, histopathological variations in the liver during aging progressed over the test period of 18 weeks between the groups according to the administration of the ENA actimineral resource A activated water were observed. Such histopathological variations were investigated by observing apotosis and damage of hepatic cells and expression of anti-activation protein using liver tissue fragments obtained from the mouse liver. As a result, with respect to all groups to which vitamin C was administrated, histopathological abnormalities were not observed, and no difference therebetween was observed. In addition, almost no TUNEL-positive cells were observed and glycogen present in the cytoplasm of normal liver cells can be identified. However, in all groups to which vitamin C was not administrated, hypertrophied hepatic stellate cells and hepatic cells exhibited a higher vacuolization than a normal level and TUNEL-positive cells were observed. Furthermore, groups to which the ENA actimineral resource A activated water was administered exhibited a significantly increased level of glycogen in the cytoplasm of hepatic cells. This result shows that the ENA actimineral resource A activated water inhibits apotosis and damage of hepatic cells caused by aging and maintains normal liver function, to maintain a normal glycogen level in liver cytoplasm and thus exhibit superior concentration-dependent hepatic cell protection effects (See FIGS. 8 to 13).

The ENA actimineral resource A activated water inhibits superoxide dismutase, an aging indicator. More specifically, as a result of tests to confirm effects of this indicator on a representative anti-aging protein, Cu,Zn-SOD, expression of Cu,Zn-SOD increases, as age of the mice increases. For the 46-week old groups, the group, to which the ENA actimineral resource A activated water was administered in a concentration of 5%, exhibited decreased expression of Cu,Zn-SOD in the liver, as compared to the excipient control group, and the decreased expression was dependent upon the concentration of activated water (See FIG. 15).

The ENA actimineral resource A activated water is present as an active ingredient in an amount of 0.001 to 15% by weight, and preferably, 0.01 to 10% by weight, based on the total weight of the pharmaceutical composition.

The daily dose of the pharmaceutical composition may be suitably controlled depending on individual variations such as age and severity of lesions, or formulations, or shape. The pharmaceutical composition is administrated twice daily in an effective amount of 0.01 to 1,000 mg for adults and may be prepared in capsules, tablets, chewing tablets, powders, dry syrups, granules, soft capsules, pills, drinks or sublingual tablets.

The pharmaceutical composition may further contain preservatives, stabilizing agents, wetting agents, emulsification promoters, pharmaceutical adjuvants such as salts or buffers to control osmotic pressure and other therapeutically effective substances. The pharmaceutical composition may be formulated for various oral or parenteral administrations by a conventional method.

Examples of formulations for oral administration include tablets, pills, hard and soft capsules, liquids, suspensions, emulsions, syrups and granules, and the like. In addition to the active ingredient, these formulations may further contain a diluting agent such as lactose, dextrose, sucrose, mannitol, sorbitol, cellulose and glycine, and a lubricant such as silica, talc, stearate, magnesium stearate, calcium stearate and polyethylene glycol. The tablet may contain a binding agent such as magnesium aluminum silicate, starch paste, gelatin, tragacanth, methyl cellulose, sodium carboxymethylcellulose and polyvinylpyrrolidone, and pharmaceutical additives including a disintegrant such as starch, agar, alginate, or sodium alginate, an absorbing agent, a coloring agent, a flavoring agent or a sweetener, if necessary. The tablet may be prepared by a conventional method such as mixing, granulation or coating. In addition, preferred parenteral formations are formulations for injection, i.e., isotonic aqueous solutions or suspensions.

However, it should be understood that an actual dose of active ingredient may be determined by various factors such as severity of symptoms, administration route selected, and age, gender, weight and health conditions of subjects.

Those skilled in the art will easily determine and prescribe an appropriate dose of the pharmaceutical composition beneficial to the skin. A daily dose of the dose according to the present invention may be varied by various factors such as disease progress of a subject, disease onset time, age, health conditions and complications. The composition composed in the weight ratio may be administrated in a dose of 1 to 500 mg/kg, preferably 30 to 200 mg/kg once or twice a day (in divided doses). The dose is not intended to limit the scope of the present invention.

In accordance with the present invention, provided is a health functional food containing the ENA actimineral resource A activated water and a sitologically acceptable additive. The health functional food is a tablet, a capsule, a pill or a liquid containing the ENA actimineral resource A activated water as an effective ingredient. The health food composition contains the ENA actimineral resource A activated water in an amount of 0.001 to 10% by weight, based on the total weight of the composition. The health food may be formulated into a drink, a caramel, a chocolate and a diet bar, or a snack using conventional ingredients such as glucose, citric acid, liquid oligosaccharide, corn syrup, soybean lecithin, butter, vegetable hardened oil, skimmed milk, sugar, margarine, edible salt, starch, wheat flour, starch syrup, maltose, sodium bicarbonate and sugar ester.

As apparent from the afore-going, the ENA actimineral resource A activated water inhibits aging-associated phenomena such as weight decrease, mortality, clinical symptoms, apotosis and damage of hepatic cells and decrease in serum vitamin C, thus being a natural substance for efficiently inhibiting or preventing in vivo aging of mammals.

The ENA actimineral resource A activated water is a natural substance capable of fundamentally protecting liver cells and thus inhibiting damage, apotosis or necrosis of hepatic cells, and potently preventing liver damage or improving liver function, without being harmful to other organs.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph comparing variations in mean weight at respective weeks between animal groups over the entire test period, after administration of ENA actimineral resource A activated water according to the present invention;

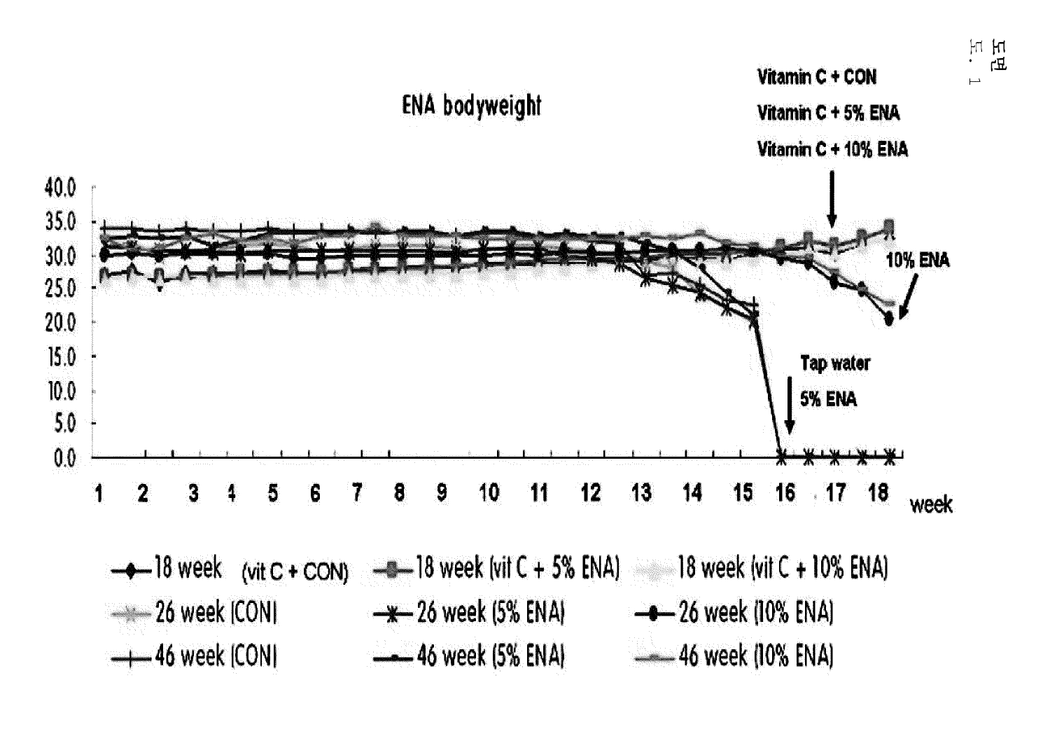

FIG. 2 is a graph showing a mean survival rate of respective groups in 18-week old mice for 18 weeks, after administration of ENA actimineral resource A activated water according to the present invention;

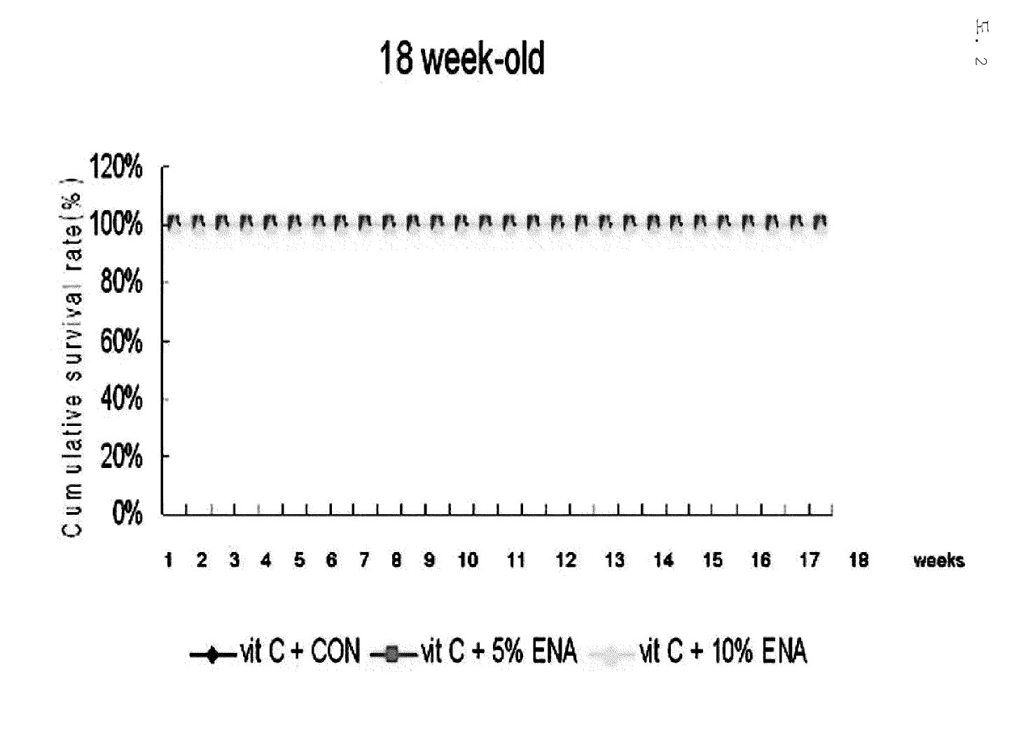

FIG. 3 is a graph showing a mean survival rate of respective groups in 26-week old mice for 18 weeks, after administration of ENA actimineral resource A activated water according to the present invention;

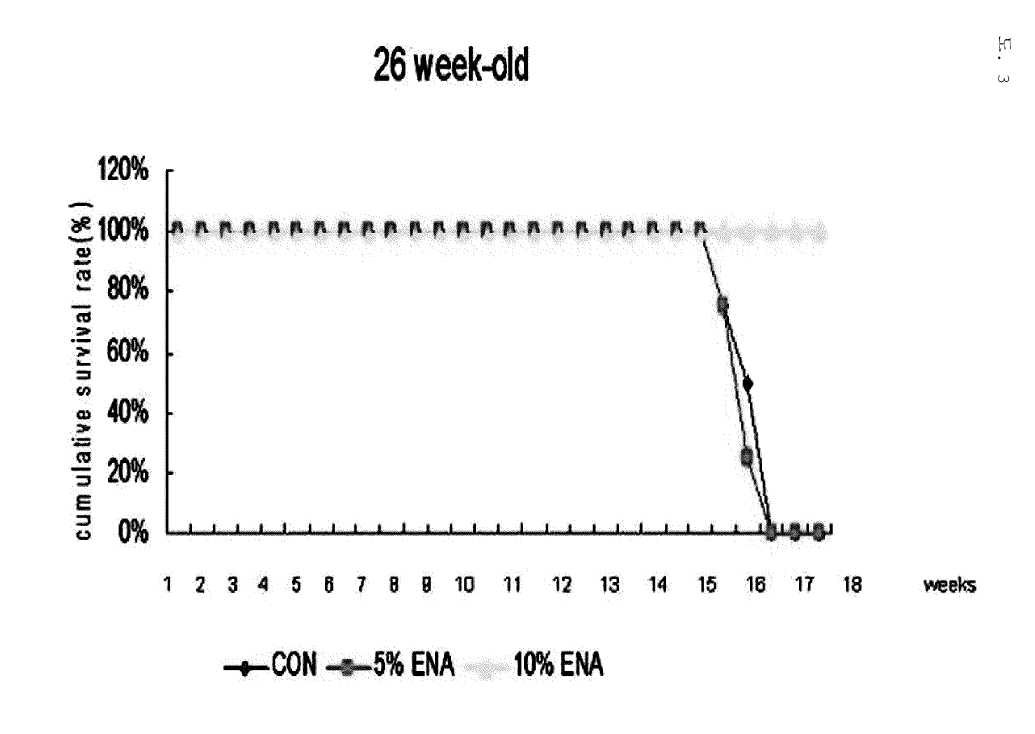

FIG. 4 is a graph showing a mean survival rate of respective groups in 48-week old mice for 18 weeks, after administration of ENA actimineral resource A activated water according to the present invention;

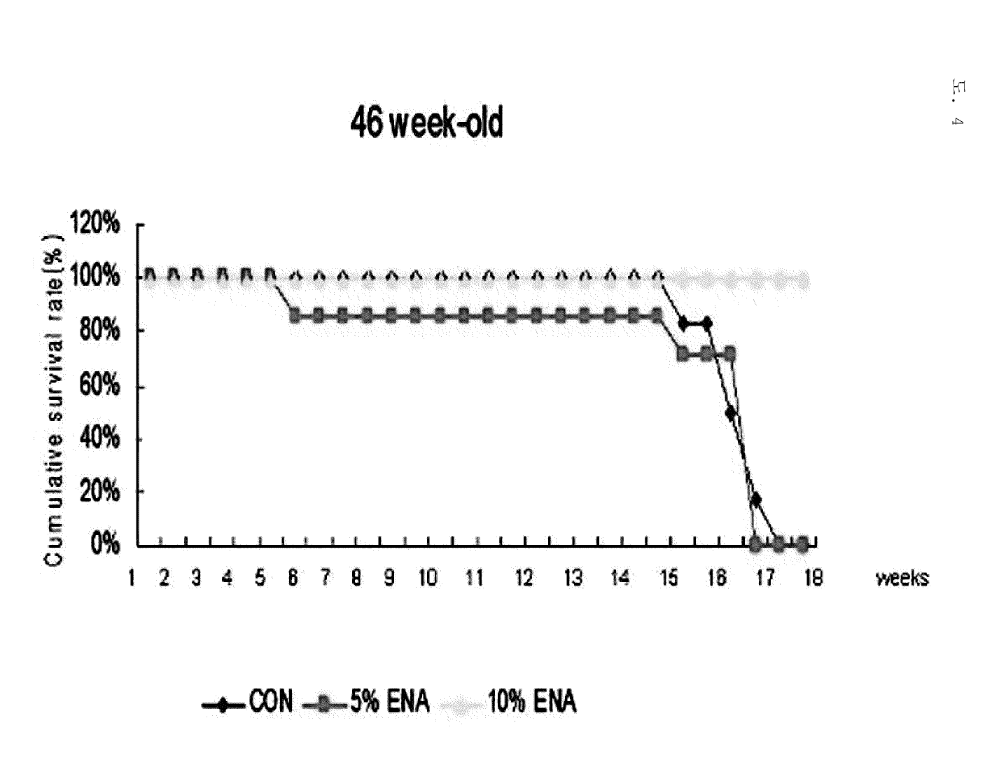

FIG. 5 is an X-ray image showing effects of ENA actimineral resource A activated water on osteogenic disorders in SMP30 Knock-out mice;

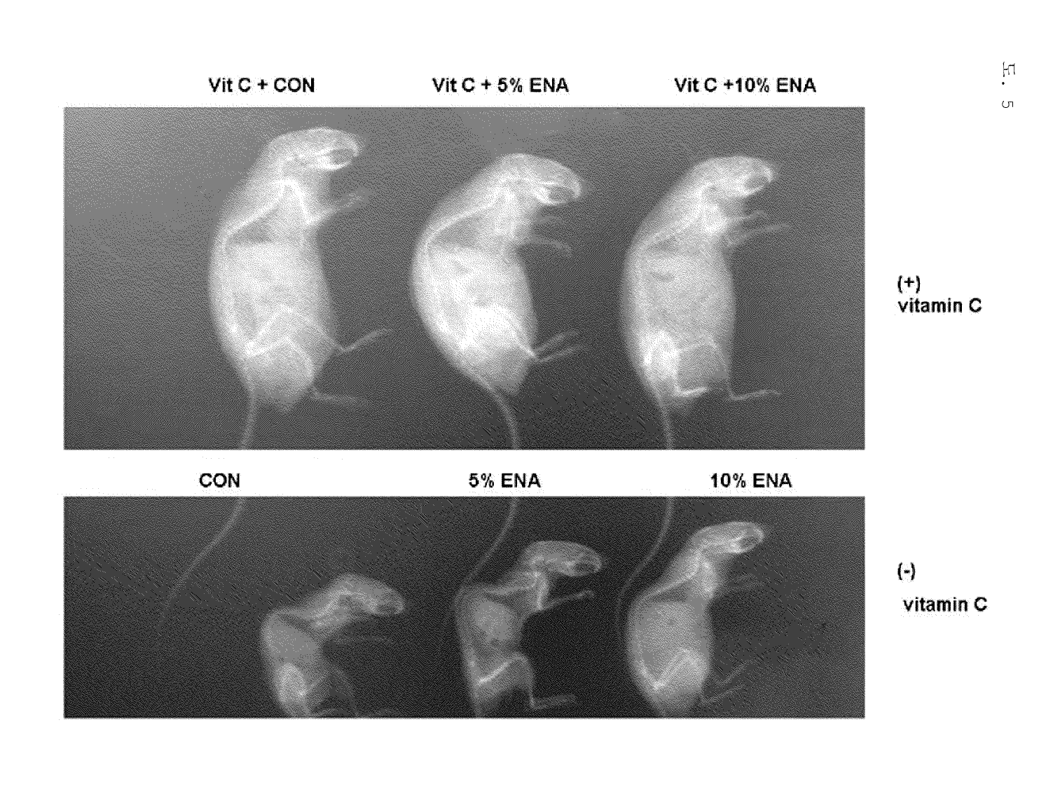

FIG. 6 is a graph showing a total vitamin C ratio in necropsied mice at 18 weeks, after administration of ENA actimineral resource A activated water according to the present invention;

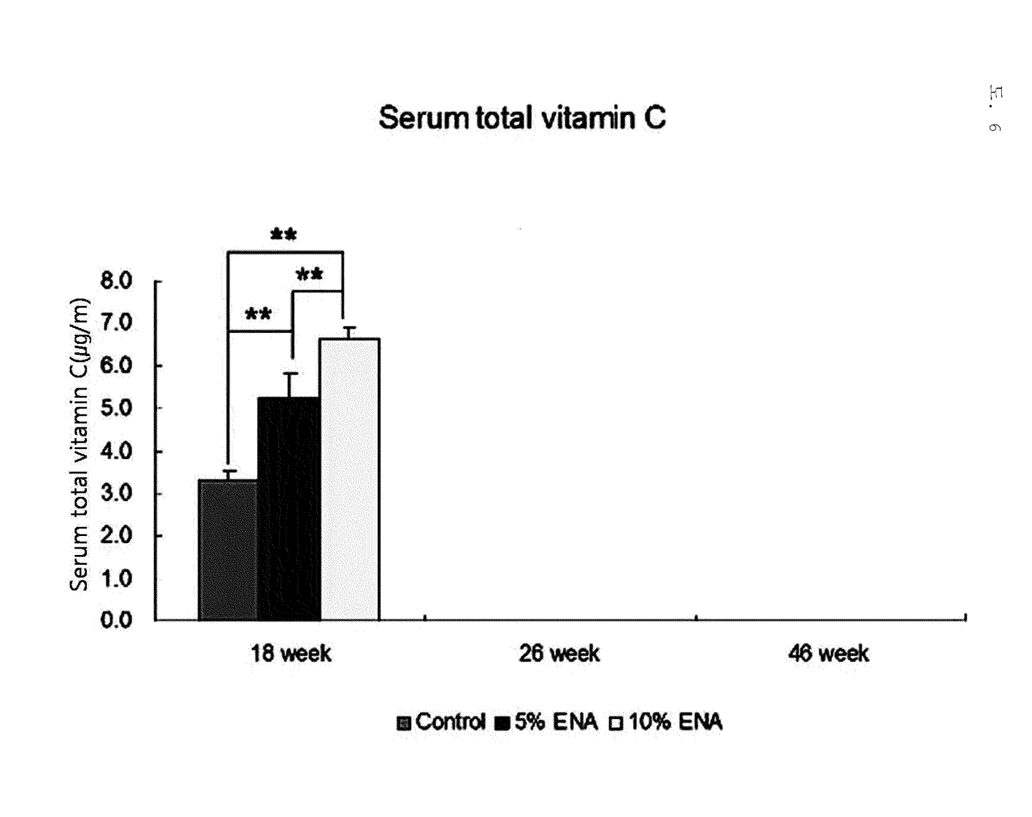

FIG. 7 is a graph showing a reductive vitamin C/oxidative vitamin C ratio in necropsied mice at 18 weeks, after administration of ENA actimineral resource A activated water according to the present invention;

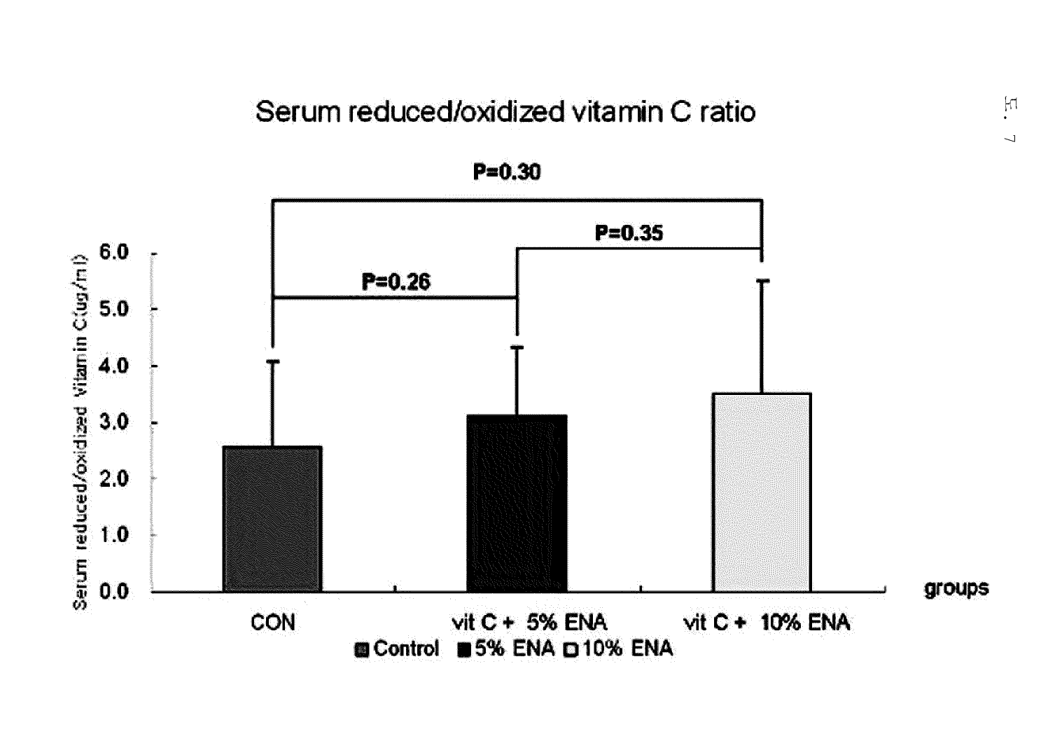

FIG. 8 illustrates histopathological analysis results of HE-positive cells with respect to the ENA actimineral resource A activated water after administration of ENA actimineral resource A activated water according to the present invention and necropsy;

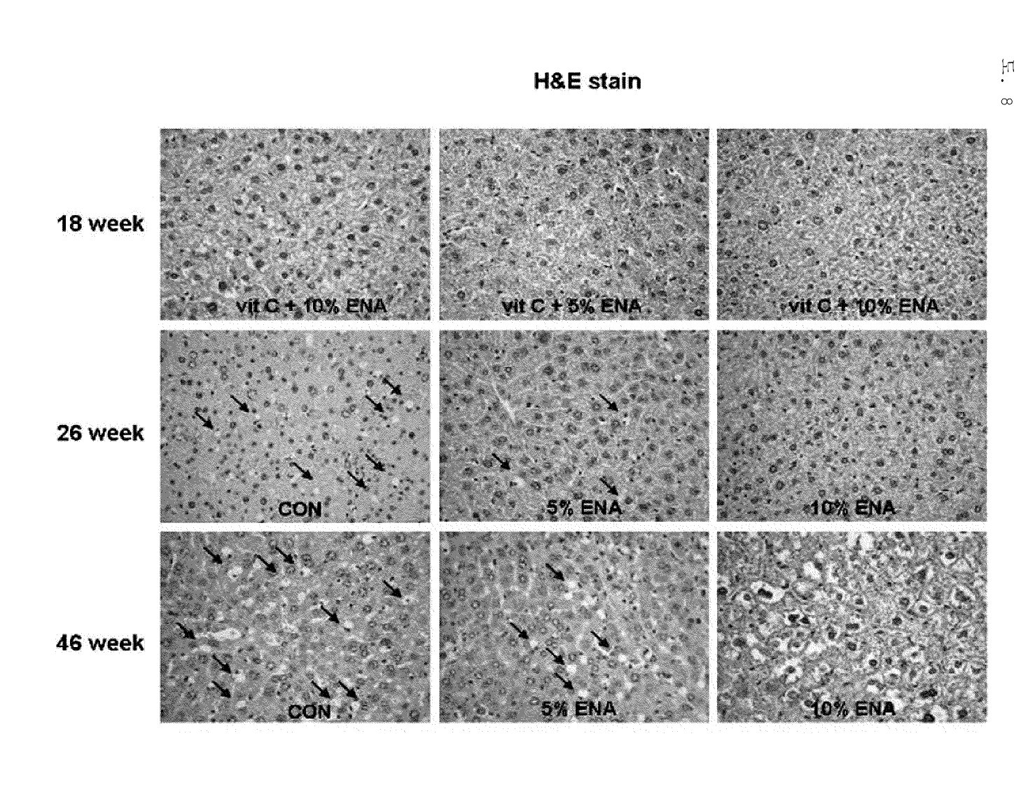

FIG. 9 illustrates comparison results of effects of the ENA actimineral resource A activated water on the number of HE-positive cells;

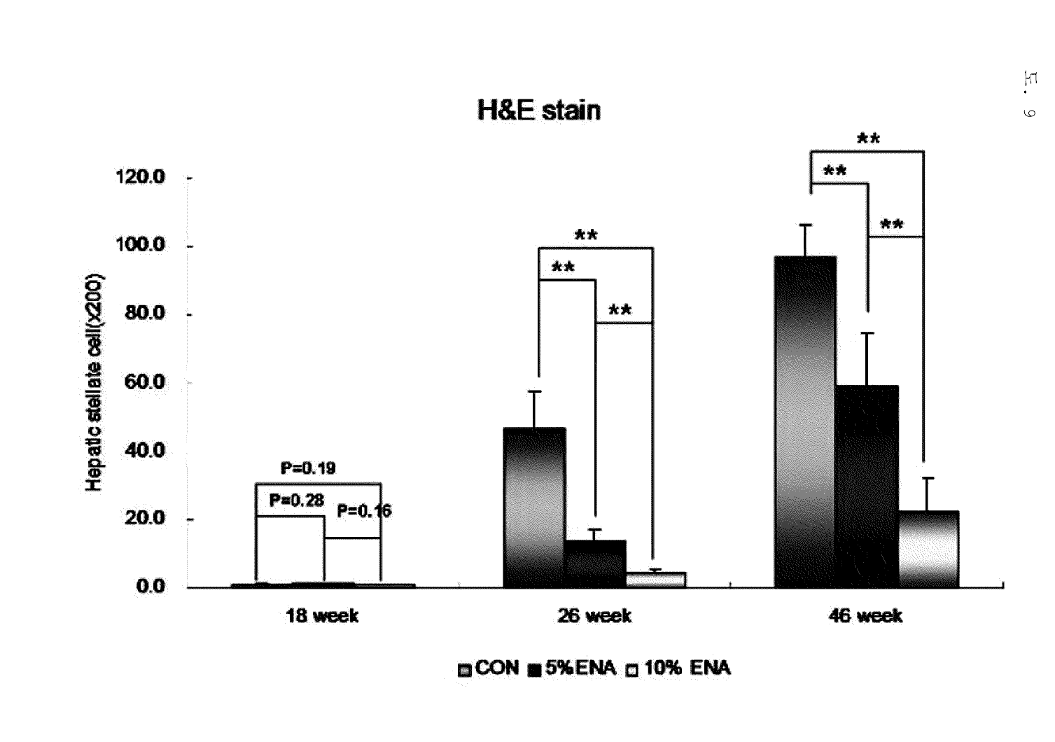

FIG. 10 illustrates histopathological analysis results of PAS-positive cells with respect to the ENA actimineral resource A activated water, after administration of ENA actimineral resource A activated water according to the present invention and necropsy;

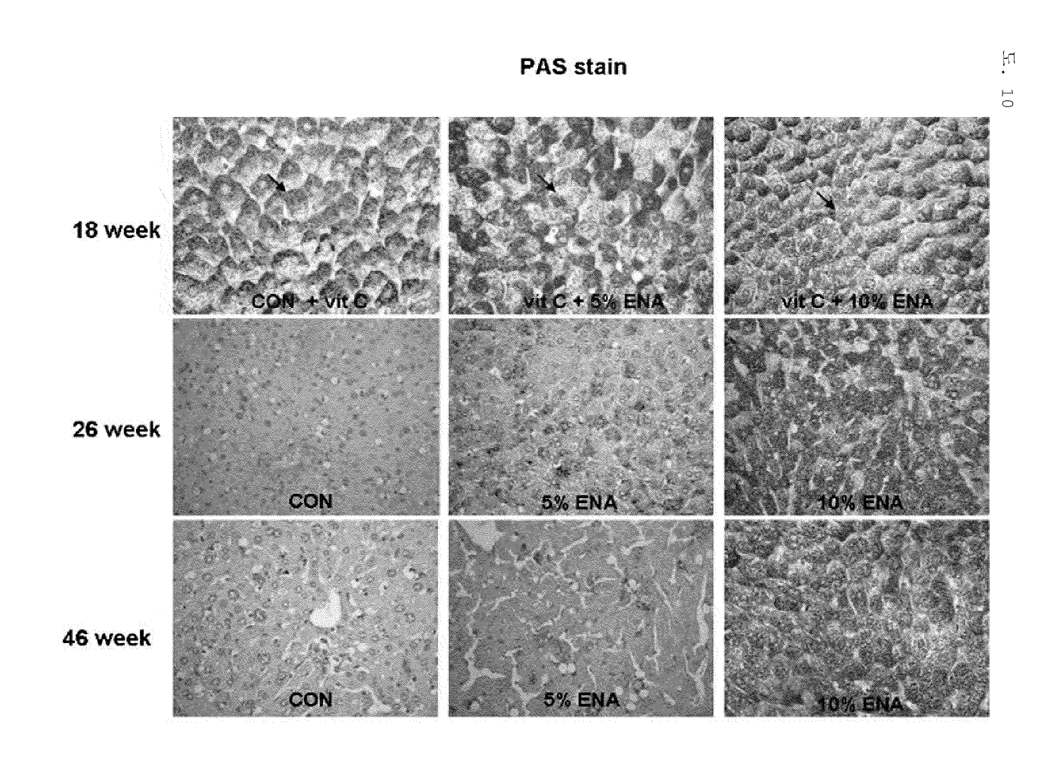

FIG. 11 illustrates comparison results of effects of the ENA actimineral resource A activated water on the number of PAS-positive cells;

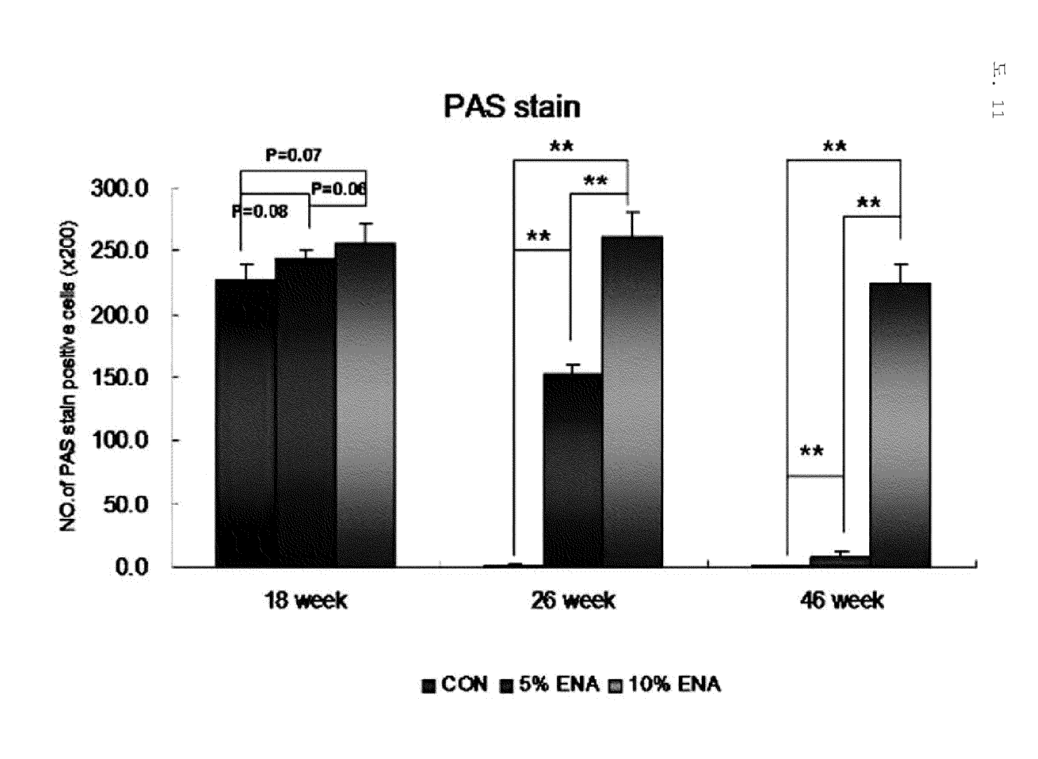

FIG. 12 illustrates comparison results of effects of the ENA actimineral resource A activated water on the number of TUNEL-positive cells on staining after administration of ENA actimineral resource A activated water according to the present invention and necropsy;

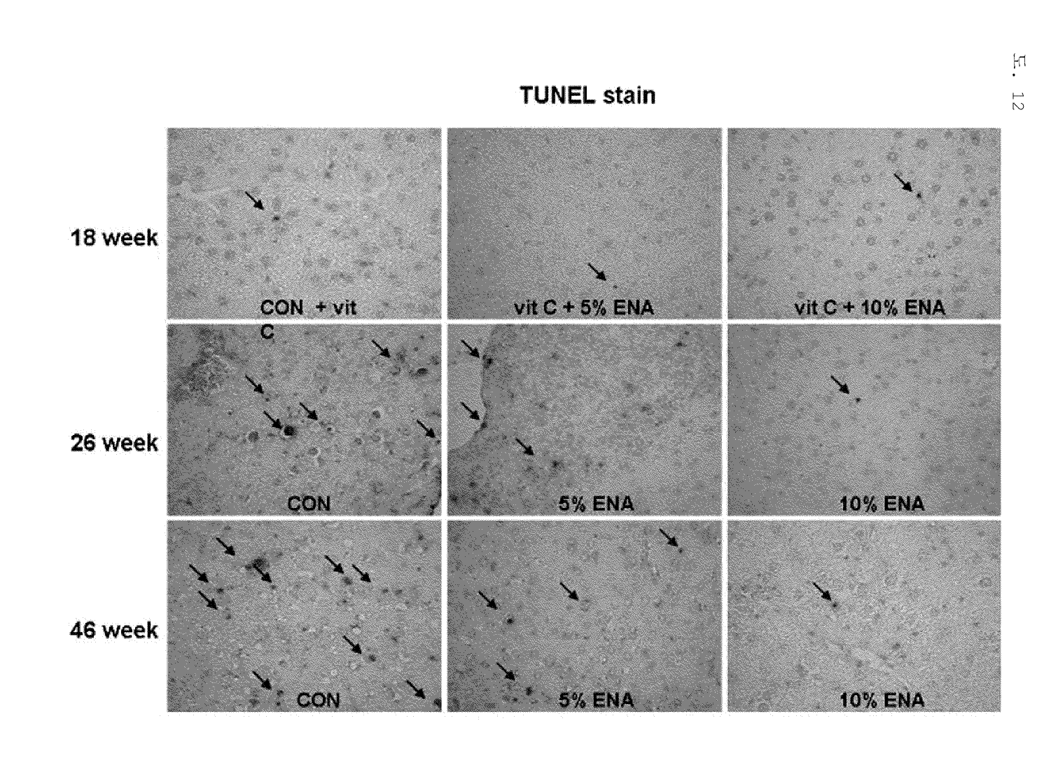

FIG. 13 illustrates comparison results of effects of the ENA actimineral resource A activated water on the number of TUNEL-positive cells;

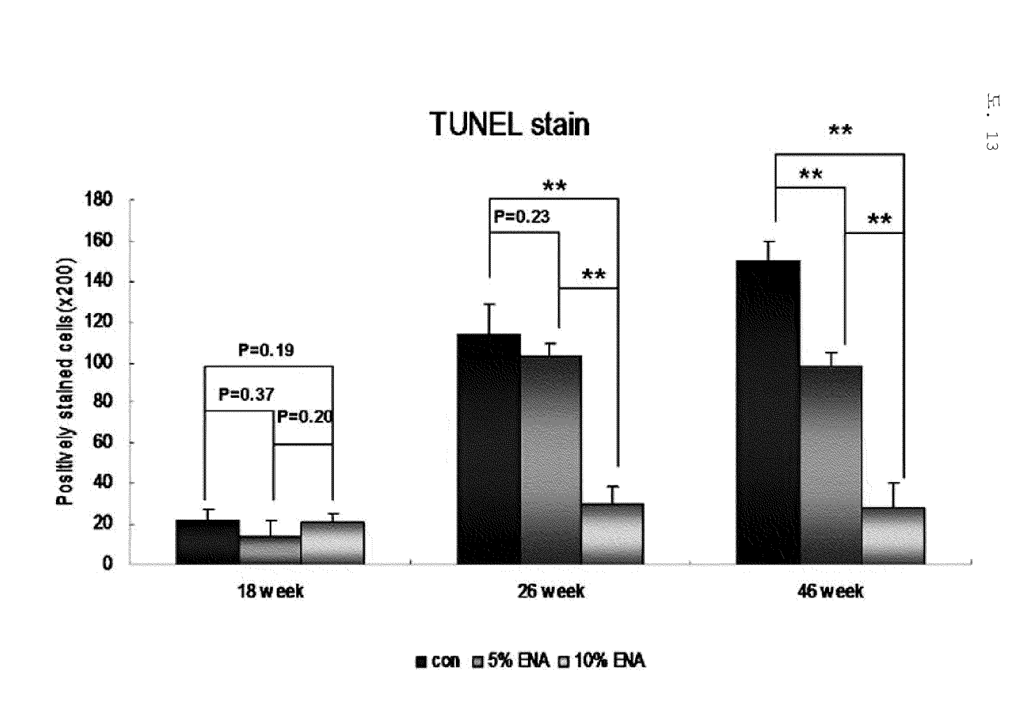

FIG. 14 illustrates histopathological analysis results confirming effects of ENA actimineral resource A activated water on expression of SMP30 in the liver, after administration of ENA actimineral resource A activated water according to the present invention; and

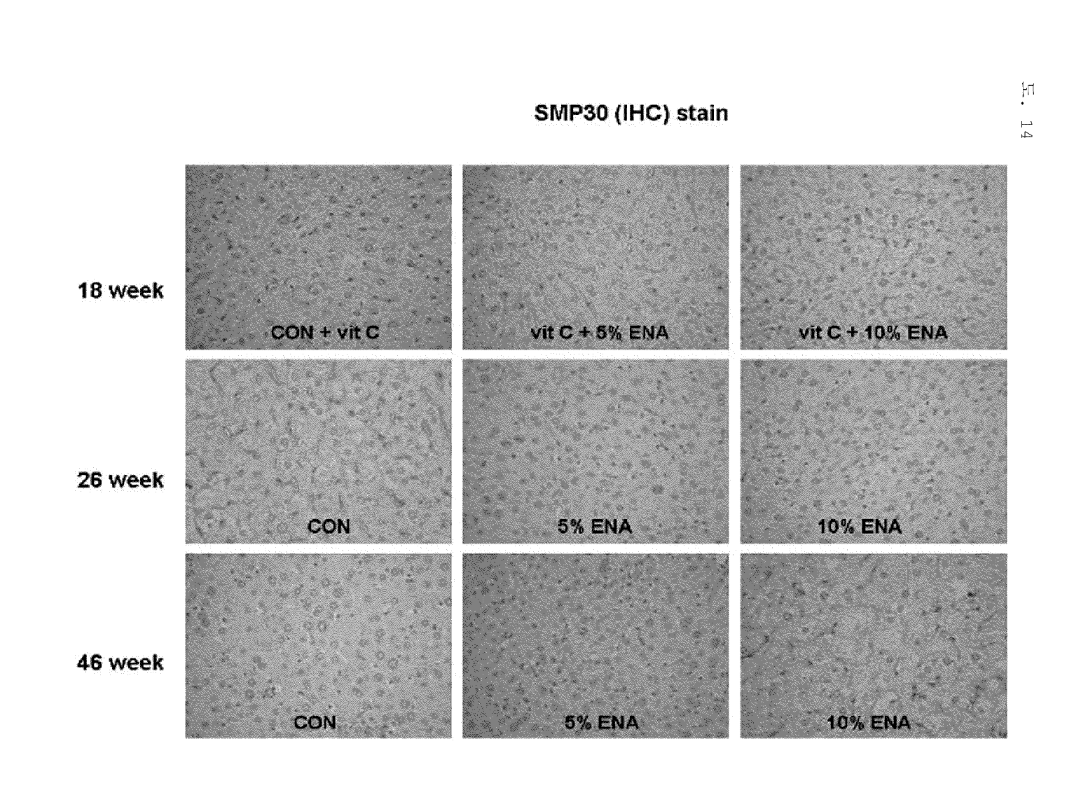

FIG. 15 illustrates comparison results confirming effects of ENA actimineral resource A activated water on expression of Cu,Zn-SOD using immunoblotting.

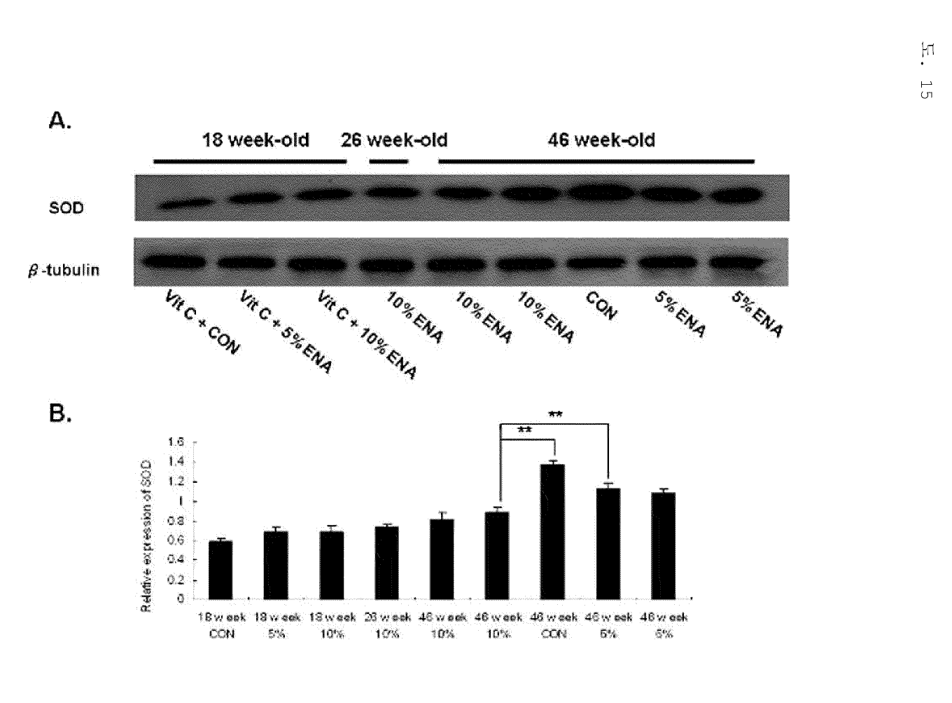

### EXAMPLES

### Preparation Example 1

### Preparation of ENA Actimineral Resource A Activated Water

A cuttlefish bone and red algae were thoroughly washed, dried and then crushed to obtain a powdery cuttlefish bone and red algae. These powdery substances were calcinated under heating at 1,000° C. for one hour. The calcinated cuttlefish bone and red algae were completely cooled to ambient temperature and then micro-powderized using a grinder. The red algae herein used was a mixture of equivalent amounts of laver (Porphyra tenera), agar (Gelidium amansii), sea string (Gracilaria verrucosa), Nemalion vermiculare, Grateloupia filicina, Gigartina tenella, Ceramium kondoi and floridean starch.

1.5 kg of the calcinated cuttlefish bone micro-powder and 4 kg of the calcinated red algae micro-powder were dissolved in 500 L of water with stirring. The resulting solution was broken at 90° C. with 10 rpm or higher of head drop using a water lifting pump for two hours to prepare an ionic solution. The ionic solution thus obtained is allowed to stand for 15 to 35 hours to naturally precipitate a mineral sludge and only the resulting clear supernatant is filtered through a filter, to prepare alkaline mineral activated water.

The mineral activated water thus obtained is referred to as an “ENA actimineral resource A activated water” and assay results of the mineral ingredients are shown in Table 1 below:

TABLE 1
Ingredient assay of ENA actimineral resource A activated water
Assay results

| Test items | Values |
|---|---|
| Iron (mg/100 g) | 0.252 |
| Calcium (mg/100 g) | 16.473 |
| Zinc (mg/100 g) | 0.100 |
| Magnesium (mg/100 g) | 0.098 |
| Sodium (mg/100 g) | 7.878 |
| Potassium (mg/100 g) | 0.953 |
| Copper (mg/100 g) | 0.012 |
| Manganese (mg/100 g) | 0.003 |
| Iodine (mg/100 g) | 1.275 |
| Phosphorus (mg/100 g) | 0.062 |
| pH | 12.85 |

As can be seen from Table 1 above, the ionic solution according to the present invention is alkaline (pH of 12.85) and contains high levels of calcium and great amounts of various metal ions.

### Examples

(1) Test Subject

1)Species and Phylogeny

Specific pathogenic organism-deficient (SPF) male 18-, 26-, 46-week old knock out C57BL/6 mice

2)Subject Origin

The mice used herein were produced by subjecting mice obtained from the Tokyo Metropolitan Institute of Gerontology (35-2 Sakae-cho, Itabashi-ku, Tokyo, 173-0015, Japan) to genetic analysis and hybridization.

3)Selection Reason of Subject

The SMP 30-knockout mice used herein underwent rapider aging than normal mice and thus acted as an useful subject of an aging test animal model and may be utilized in test result analysis.

4) Quarantine and Taming

SMP30 knockout mice were prepared by cross-breeding 10 male and 20 female SMP30 KO C57BL/6 mice obtained from the Tokyo Metropolitan Institute of Gerontology in the department of laboratory animal medicine, college of veterinary medicine, Kyungpook National University. Only SMP30 knockout mice were selected from F1 mice born by cross-breeding through tail DNA genotype analysis using PCR.

5)Genotype Analysis of Animals

Genotype analysis of animals was carried out by tail DNA genotype analysis using PCR. Genomic DNA was extracted from the tail in mice in accordance with the composite method disclosed in the literature. The mouse tail was subjected to biopsy and frozen at −80° C. for at least 15 minutes. Then, 300 mL of a lysis buffer (60 mL Tris-HCl pH 8.0; 500 mL EDTA; 10% SDS; 0.2 mg/ml ribonuclease A; 1 mg/ml proteinase K) was added to each sample. Sample lysis was carried out by reacting the mixture, while vibrating in a CO₂ incubator at 56° C. for 5 hours. After lysis, each sample was centrifuged at 13,000 rpm at ambient temperature for 10 minutes, to remove tissue residues. Then, the supernatant was isolated and 500 mL of isopropanol was added thereto to precipitate genomic DNA. The resulting solution was washed with ethanol. The sample was centrifuged for 10 minutes. After removal of the supernatant, the precipitated pellet was dried at ambient temperature. The pellet was dissolved in 50 uL of 5 mM Tris-HCl buffer (pH 8.5) and allowed to stand at 65° C. for 5 minutes. The genomic DNA was quantitized using a spectrometer (Backman, Fullerton, USA), diluted to a level of 250 ng/ul and 1 uL of a PCR mixture was added to the diluted DNA. Knockout confirmation was carried out using primers TA4 (5′-CAAGTAACTCTAGGTATGGAC-3′), TS3 (5′-CTAGCCATGGTGGATGAAGAT-3′) and NEO (5′ -TCGTGCTTTACGGTATCGCCGCTCCCGATT-3′).

(2) Breeding Environment

1) Environment

The mice used herein were tamed and bred in an automatic constant temperature and humidity regulator wherein temperature is 22±3° C., relative humidity is 50±10%, illumination period is 12 hours (lighting at 8 AM and lights-out at 8 PM) in the department of laboratory animal medicine, college of veterinary medicine, Kyungpook National University. Variations in breeding conditions affecting test results were not accepted during the overall test period.

2) Breeding Box, Density and Identification of Breeding Box

5 mice were placed in each polycarbonate breeding box (240 W×390 L×175 Hmm) during the test period. Subject identification was performed by tail-marking and ID card marking for each breeding box using a permanent marker

3) Feed and Water

a) Method for Supplying Feed

A solid feed for test animals (PMI Nutrition International, 505 North 4^{th} Street Richmond, Ind. 47374, USA) as the feed was sterilized by irradiation (13.2 kGy) and then freely provided.

b) Method for Supplying Water

Water was freely provided to mice using a water bottle containing tap water.

c) Constitution and Administration of Experimental Group

Experiments were divided into Experiment Plans A and B. Experiment Plan A utilized 18-week old male SMP30 knock-out C57BL/6 mice and was divided into three groups (n=3 for each group), Control group, Experimental Group 1 and Experimental Group 2. Experiment Plan B utilized 26-week and 46-week old male SMP30 knock-out C57BL/6 mice and was divided into three groups (n=3 for each group), Control group, Experimental Group 1 and Experimental Group 2, as in Experimental Plan 1. The 26-week old mice were divided into three groups (n=4 for each group) and 46-week old mice were divided into three groups (n=6 for each group). The ENA actimineral resource A activated water herein used was a diluted solution (about 5% and 10%) of the crude activated water obtained in Preparation Example 1 in tap water. The diluted solution was freely fed to the mice over the test period of 18 weeks. At 18 weeks, all test animals were subjected to autopsy, and blood and organ samples thereof were collected for histopathological examination.

| | | | Number of | |
|---|---|---|---|---|
| | Groups | Animal Type | subject | Treatment |
| Experiment | Control | SMP30 KO mice | N = 8 | |
| Plan A | group | (18-week) | | |
| | 5% ENA | SMP30 KO mice | N = 8 | |
| | | (18-week) | | |
| | 10% ENA | SMP30 KO mice | N = 8 | |
| | | (18-week) | | |
| Experimental | Control | SMP30 KO mice | N = 4 | Potable tap |
| Plan B | group | (26-week) | | water |
| | | SMP30 KO mice | N = 6 | |
| | | (46-week) | | |
| | 5% ENA | SMP30 KO mice | N = 4 | 5% ENA |
| | | (26-week) | | |
| | | SMP30 KO mice | N = 7 | |
| | | (46-week) | | |
| | 10% ENA | SMP30 KO mice | N = 4 | 10% ENA |
| | | (26-week) | | |
| | | SMP30 KO mice | N = 7 | |
| | | (46-week) | | |

* ENA: ENA actimineral resource A activated water

* During test period, potable water was made freely available

(4) Items of Observation and Test Examination

1) Effects of Sample on Weight Variations

Male SMP 30-knockout mouse aging models were weighted weekly over the test period of 12 weeks and variations in weight were observed. The results thus obtained are shown in Table 1 below.

2) Effects of Sample on Aging-Associated Clinical Symptoms

Effects of sample on animal skeleton variations and physical conversion in male SMP 30-knockout mouse aging models were evaluated over the test period of 12 weeks in order to observe naturally-occurring aging-associated clinical symptoms.

3) Effects of Sample on Survival Rate

Variations and difference in survival rate between groups in male SMP 30-knockout mouse aging models were observed by X-ray irradiation over the entire test period of 18 weeks. The results thus obtained are shown in Table 1 below.

TABLE 2
Effects of ENA actimineral resource A activated water on
survival rate associated with anti-aging activity
Variations in survival rate
Study: path200403 Number of groups: n = 9 Gender: male
Dose: ENA actimineral resource A activated water
Survival rate

| Group I.D. | 6 weeks | 10 weeks | 14 weeks | 18 weeks |
|---|---|---|---|---|
| 1 | 8/8 | 8/8 | 8/8 | 8/8 |
| 2 | 8/8 | 8/8 | 8/8 | 8/8 |
| 3 | 8/8 | 8/8 | 8/8 | 8/8 |
| 4 | 4/4 | 4/4 | 2/4 | 0/4 |
| 5 | 4/4 | 4/4 | 2/4 | 0/4 |
| 6 | 4/4 | 4/4 | 4/4 | 4/4 |
| 7 | 6/6 | 6/6 | 3/6 | 0/6 |
| 8 | 7/7 | 6/7 | 5/7 | 0/7 |
| 9 | 7/7 | 7/7 | 7/7 | 7/7 |

4) Effects of Sample on Serum Vitamin C

In order to confirm effects of sample on serum vitamin C in male SMP 30-knockout mouse aging models over the test period of 18 weeks, 100 uL of serum isolated from blood samples by centrifugation (3,000 g, 15 minutes) was treated with 450 mL of 3% metaphosphoric acid and the resulting mixture was centrifuged at 10,000g and 4° C. for 10 minutes, 90 mL of the supernatant was mixed with 16.4 uL of 0.1% dithiothreitol (DTT), the resulting mixture was allowed to stand in an ice bath for 30 minutes, and 957.6 uL of 3% metaphosphoric acid was added thereto. The resulting mixture was centrifuged at 1,000g and 4° C. for 10 minutes, a level of vitamin C in the blood was measured by high performance liquid chromatography (HPLC) using a Shodex-5SIL-4E column (4.6 250 mm; Showa Denko, Tokyo).

5) Effects of Sample on Pathological Variations by Aging in the Liver

In order to confirm effects of sample on pathological variations in the liver of male SMP 30-knockout mouse aging models during aging over the test period of 18 weeks, hematoxylin and eosin staining, periodic acid staining, TUNEL assay staining and immunohistochemistry were performed. The stains were observed with an optical spectroscope and cells exhibiting positive reaction to each stain were calculated to observe differences in apotosis and damage of cells, and expression of anti-oxidation proteins between groups. All pathological monitoring was carried out using a double screen. The results thus obtained are shown in Table 3 below.

TABLE 3
Lesions and damage of liver in different ages (week) and groups of mice

| | | | Mean No. of |
|---|---|---|---|
| | | | hypertrophic hepatic |
| Age | | | stellate cells/field |
| (week) | Groups | Liver disorder | (x 100) |
| 18 | Control | Normal finding | 1.1 ± 0.3 |
| | group | | |
| | 5% ENA | Normal finding | 1.3 ± 0.3 |
| | 10% ENA | Normal finding | 0.8 ± 0.3 |
| 26 | Control | Apotosis, necrosis and vacuole | 46.8 ± 10.9 |
| | group | variations in hepatic cells, and | |
| | | hypertrophy and hyperplasia of | |
| | | hepatic stellate cell | |
| | 5% ENA | Apotosis, necrosis and vacuole | 13.8 ± 3.4 |
| | | variations in hepatic cells, and | |
| | | hypertrophy and hyperplasia of | |
| | | hepatic stellate cell | |
| | 10% ENA | Apotosis, necrosis and vacuole | 4.4 ± 1.1 |
| | | variations in hepatic cells, and | |
| | | hypertrophy and hyperplasia of | |
| | | hepatic stellate cell | |
| 46 | Control | Apotosis, necrosis and vacuole | 96.8 ± 9.6 |
| | group | variations in hepatic cells, and | |
| | | hypertrophy and hyperplasia of | |
| | | hepatic stellate cell | |
| | 5% ENA | Apotosis, necrosis and vacuole | 59.2 ± 15.5 |
| | | variations in hepatic cells, and | |
| | | hypertrophy and hyperplasia of | |
| | | hepatic stellate cell | |
| | 10% ENA | Apotosis, necrosis and vacuole | 22.0 ± 10.2 |
| | | variations in hepatic cells, and | |
| | | hypertrophy and hyperplasia of | |
| | | hepatic stellate cell | |

ENA: ENA actimineral resource A activated water

6) Effects of Sample on Expression of Anti-Oxidation Proteins in the Liver

In order to confirm effects of sample on expression of the representative anti-oxidative protein, superoxide dismutase (Cu,Zn-SOD) after the test period of 18 weeks in male SMP 30-knockout mouse aging models, the liver tissue frozen at −70° C. was homogenized in a RIPA buffer containing 0.1 mM sodium orthovanadate (Na₃Vo₄) and protease inhibitor cocktail tablet (Roche, Mannheim, Germany). The resulting liver sample was centrifuged at 4° C. and 4,000 rpm for 10 minutes to remove lipids. The resulting supernatant was centrifuged at 4° C. and 14,000 rpm for 20 minutes again to obtain a supernatant. A level of protein in the supernatant was measured by protein quantitative assay (Bradford method). The protein sample (80 ug/well) was subjected to 10% SDS-polyacrylamide gel electrophoresis. Proteins in the electrophorized gel were electro-transferred through a PVDF membrane (Schleicher & Schuell, Dassel, Germany) for specific protein detection (immunblotting). Then, the protein sample was blocked in a blocking solution (wherein 3% bovine serum albumin was dissolved in Tris-buffered saline) for one hour and then reacted with Cu,Zn-SOD (1:100, Stressgen, Victoria, Canada) and β-tubulin (1:1000, Sigma, Mo., USA). The resulting sample was thoroughly washed with a TBS buffer solution containing 0.5 Twin 200 and then reacted with a diluted solution (at a ratio of 1:1000 to 1:2000) of a secondary antibody, corresponding to a primary antibody, at ambient temperature for one hour. The sample was thoroughly washed with a TBS buffer solution again, reacted with a Super Signal West Dura Extended Duration Substrate (PIERCE, Ill., USA) to observe a specific reaction, and then exposed to a medical X-ray film (Kodak, Tokyo, Japan).

(5) Statistical Method

Statistical analysis of data thus obtained was carried out using a paired (not unpaired) T-test to evaluate difference in mean between two groups. T-test is a statistical hypothesis test wherein difference in mean between two groups is standardized with variance of the groups and the resulting value is statistically analyzed. T-test is divided into two cases, i.e., one case wherein the groups have identical variance and other case wherein the groups have different variances. This analysis was carried out using a statistical program, GraphPad inStat (version 3.05, GraphPad Software Inc.). The significance levels of testing were 5% and 1%.

[Pharmaceutical Composition Formulation 1] Tablet

A mixture of 80 mg of ENA actimineral resource A activated water, 200 mg of galacto oligosaccharide, 60 mg of lactose and 140 mg of maltase was granulated using a fluidized bed dryer, 6 mg of sugar ester was added to the granules and the resulting mixture was tabletted using a tablet press. The total weight of tablet ingredients was 600 mg.

[Health Food Composition Formulation 2] Drink

300 mL of distilled water was added to a mixture of 80 mg of the ENA actimineral resource A activated water, 10 mg of glucose, 0.6 g of citric acid and 25 g of liquid oligosaccharide and the resulting mixture was filled in an amount of 200 mL in each bottle and then sterilized at 130° C. for 4 to 5 seconds to prepare a drink.

What is claimed is:

1. A pharmaceutical composition comprising an alkaline aqueous solution as an active ingredient, for preventing aging due to inhibitory activity on decrease in serum vitamin C, or preventing liver damage or improving liver function due to inhibitory activity on damage, apotosis or necrosis of hepatic cells,
wherein the alkaline aqueous solution is prepared according to the following steps:
crush-powderizing one or more selected from Sepia bone (Sepia esculenta), laver (Porphyra tenera), agar (Gelidium amansii), sea string (Gracilaria verrucosa), Nemalion vermiculare, Grateloupia filicina, Gigartina tenella, Ceramium kondoi and floridean starch;
calcinating the powdery substance under heating at 1,000 to 2,000° C.;
cooling and micro-powderizing the substance, and adding the micro-powdery substance to water of 80 to 100° C.;
breaking the resulting mixture with a head drop at 10 rpm or higher using a water lifting pump to prepare an ionic solution; and
filtering the ionic solution to obtain an alkaline aqueous solution.

2. The pharmaceutical composition according to claim 1, wherein the ENA actimineral resource A activated water is present in an amount of 0.001 to 15% by weight, based on the total weight of the pharmaceutical composition.

3. The pharmaceutical composition according to claim 2, wherein the pharmaceutical composition is formulated into a capsule, a tablet, a chewing tablet, a powder, a dry syrup, a granule, a soft capsule, a pill, a drink or a sublingual tablet.

4. A health food or health supplement comprising an alkaline aqueous solution as an active ingredient, for preventing aging due to inhibitory activity on decrease in serum vitamin C, or preventing liver damage or improving liver functions due to inhibitory activity on damage, apotosis or necrosis of hepatic cells,
wherein the alkaline aqueous solution is prepared by the following steps:
crush-powderizing one or more selected from Sepia bone (Sepia esculenta), laver (Porphyra tenera), agar (Gelidium amansii), sea string (Gracilaria verrucosa), Nemalion vermiculare, Grateloupia filicina, Gigartina tenella, Ceramium kondoi and floridean starch;
calcinating the powdery substance under heating at 1,000 to 2,000° C.;
cooling and micro-powderizing the substance, and adding the micro-powdery substance to water of 80 to 100° C.;
breaking the resulting mixture with a head drop at 10 rpm or higher using a water lifting pump to prepare an ionic solution; and
filtering the ionic solution to obtain an alkaline aqueous solution.

5. The health food or health supplement according to claim 4, wherein the health food or the health supplement is formulated into a drink, a caramel, a chocolate, a diet bar or a snack.
